(19) United States
(12) Patent Application Publication
(10) Pub. No.: US 2004/0102753 A1
(43) Pub. Date: May 27, 2004

(54) Male incontinence absorbent article and method of producing same

(75) Inventor: Amanda R. Butler, Woodstock, GA (US)

Correspondence Address: SCHWEGMAN, LUNDBERG, WOESSNER & KLUTH, P.A., P.O. BOX 2938, MINNEAPOLIS, MN, 55402, US

(73) Assignee: Kimberly-Clark Worldwide, Inc.

(21) Appl. No.: 10/305,521

(22) Filed: Nov. 26, 2002

(51) Int. Cl. A61F 13/15

(52) U.S. Cl. 604/385.01

(57) ABSTRACT

The present invention provides a male incontinence absorbent article comprising an absorbent sheet having a body-side and a garment-side, the absorbent sheet folded to form a central section and two side sections, each of the two side sections and central section having an inner surface and an outer surface; and an intake/distribution layer covering the inner surface and the outer surface of each of the two side sections to form covered side sections, wherein the covered side sections form an opposed pair of longitudinal flaps. The longitudinal flaps are optionally unfolded into an open position during use. The present invention also provides a method for making the male incontinence absorbent article.

### FIELD

The present invention relates to absorbent articles, and, in particular, to a male incontinence absorbent article and method of producing same.

### BACKGROUND

Male incontinence absorbent articles are known in the art. Such articles are designed to be worn inside undergarments, often secured to the undergarment with releasable adhesive. However, male genitalia does not typically stay centered on such products during movement, leading to urine insults along the edges.

Attempts to overcome this problem include providing articles having elastic barrier cuffs. Such articles, however, can be uncomfortable and constrictive.

Therefore, there is a need in the art to provide a comfortable and non-constrictive male incontinence absorbent article with improved leakage control.

### SUMMARY

The invention provides an absorbent article comprising an absorbent sheet having a body-side and a garment-side, the absorbent sheet folded to form a central section and two side sections, each of the two side sections and central section having an inner surface and an outer surface; and an intake/distribution layer covering the inner surface and the outer surface of each of the two side sections to form covered side sections, wherein the covered side sections form an opposed pair of longitudinal flaps.

In one embodiment, the opposed pair of longitudinal flaps are optionally unfolded into an open position during use, thus serving as side shields in the unfolded position, further wherein the inner surface of each of the two side sections is the body-side.

In another embodiment, the longitudinal flaps are flat-folded during use, thus creating an additional absorbent layer substantially covering the covered central section when flat-folded, further wherein the outer surface of the two side sections is the body-side.

The invention further includes a method of making an incontinence article comprising layering together an intake/distribution layer, a body-side liner and a first absorbent layer. The layers are then folded such that the intake/distribution layer and body-side liner cover all surfaces that may come in contact with a user's body. The end portions of the folds, i.e., the portions containing only the body-side liner, are then optionally bonded together. The process continues by combining the folded layers with a second absorbent layer and a substantially moisture impermeable component to form an absorbent male incontinence article. In one embodiment, the folded layers have a C-folded cross-section. In one embodiment some or all of the folded layers, such as the intake/distribution layer and first absorbent layer, are bonded together prior to being folded. In another embodiment, the first absorbent layer is wrapped in a thin tissue material to help maintain the integrity of the first absorbent layer during manufacturing.

The present invention provides optional extra protection against leakage off the sides using soft and comfortable side shields made of absorbent material that helps to contain the penis within the product's center or target absorbency zone, thus absorbing urine on all sides. The ability, however, to keep the side shields folded into the center of the product during use provides the consumer with added flexibility in determining the most appropriate configuration for his personal needs.

### BRIEF DESCRIPTION OF THE DRAWINGS

FIG. 1 is a simplified cut-away top view of a male incontinence absorbent article having side sections in a closed position in one embodiment of the present invention.

FIG. 2 is a simplified cross-sectional view of a male incontinence absorbent article in an open position in one embodiment of the present invention.

FIG. 3 is a simplified cut-away top view of a male incontinence absorbent article having side sections in an open position in one embodiment of the present invention.

FIG. 4 is a simplified perspective view of a male incontinence absorbent article in one embodiment of the present invention.

FIG. 5 is a block diagram describing a method of making a male incontinence absorbent article in one embodiment of the present invention.

### DETAILED DESCRIPTION

In the following detailed description of the preferred aspects, reference is made to the accompanying drawings, which form a part hereof, and in which is shown by way of illustration specific preferred aspects in which the invention may be practiced. These aspects are described in sufficient detail to enable those skilled in the art to practice the invention, and it is to be understood that other aspects may be utilized and that chemical, mechanical, procedural and other changes may be made without departing from the spirit and scope of the present invention. The following detailed description is, therefore, not to be taken in a limiting sense, and the scope of the present invention is defined only by the appended claims, along with the full scope of equivalents to which such claims are entitled.

Various definitions used throughout the specification are provided first, followed by a description of various embodiments of the present invention.

Definitions

As used herein, the term “disposable absorbent article” refers to an article that is disposed of after use, i.e., is not intended to be washed and reused. Such an article is a layered material typically comprising a substantially moisture impermeable garment-side (outer) cover, an absorbent assembly comprised of one or more absorbent layers, an intake/distribution layer and a moisture permeable body-side (inner) liner.

As used herein, the term “nonwoven web” means a structure or a web of material that has been formed without use of traditional fabric forming processes, such as weaving or knitting, to produce a structure of individual fibers or threads that are intermeshed, but not in an identifiable, repeating manner. Non-woven webs can be formed by a variety of conventional processes such as, for example, meltblowing processes, spunbonding processes, film aperturing processes and staple fiber carding processes.

As used herein, the terms “upper,” “lower”, “inner” and “outer” are used to indicate the direction relative to the user wearing an absorbent garment over the crotch region. For example, the term “inner” refers to a “body-side,” i.e., side closest to the body of the user, while the term “outer” refers to a “garment-side,” i.e., side furthest away from the body of the user. The term “upper” refers to a portion of the garment that is higher up on the user, i.e., towards the user's head, as compared with a portion that is “lower,” i.e., further away from the user's head.

As used herein, the term “longitudinal” means of or relating to length or the lengthwise direction, and in particular, the direction running between the upper and lower portions of the garment.

As used herein, the term “lateral,” means situated on, directed towards or running from side to side.

### DESCRIPTION OF THE EMBODIMENTS

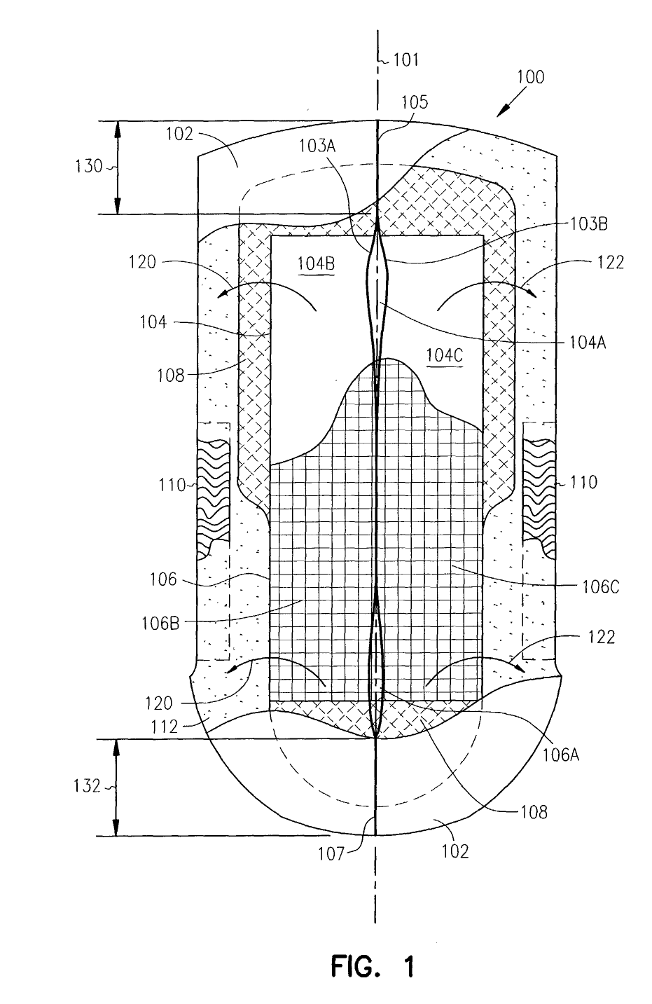

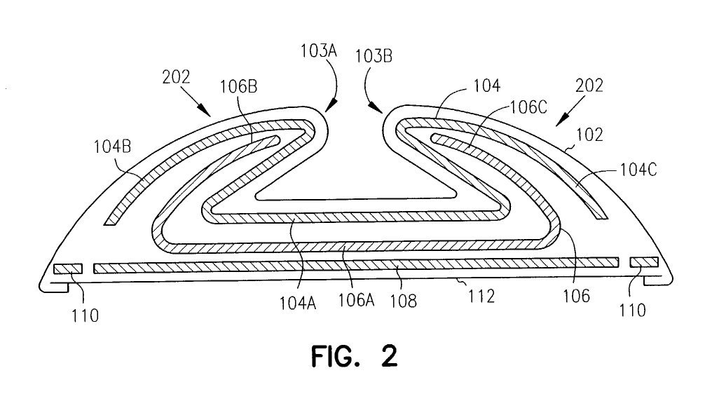

The disposable absorbent article of the present invention is specifically designed to address urinary incontinence and be compatible with the adult male anatomy. In the embodiment shown in FIG. 1, the article 100 comprises a moisture permeable body-side (inner) liner (hereinafter “liner”) 102, absorbent components, including a C-folded absorbent sheet 106 and a garment-side absorbent layer 108, an intake/distribution layer 104 and a substantially moisture impermeable garment-side (outer) cover (hereinafter “cover”) 112. The article further contains side elastics 110 as shown. In this embodiment, the intake/distribution layer 104 is a substantially rectangular component that surrounds the body-side surfaces of the substantially rectangular C-folded absorbent sheet 106 (see FIG. 2). The intake/distribution layer 104 is also in fluid communication with both the C-folded absorbent sheet 106 and the garment-side absorbent layer 108, which has a “paddle” shape in this embodiment. The arrangement of the above-described layers can be seen most clearly in the cross-sectional view shown in FIG. 2.

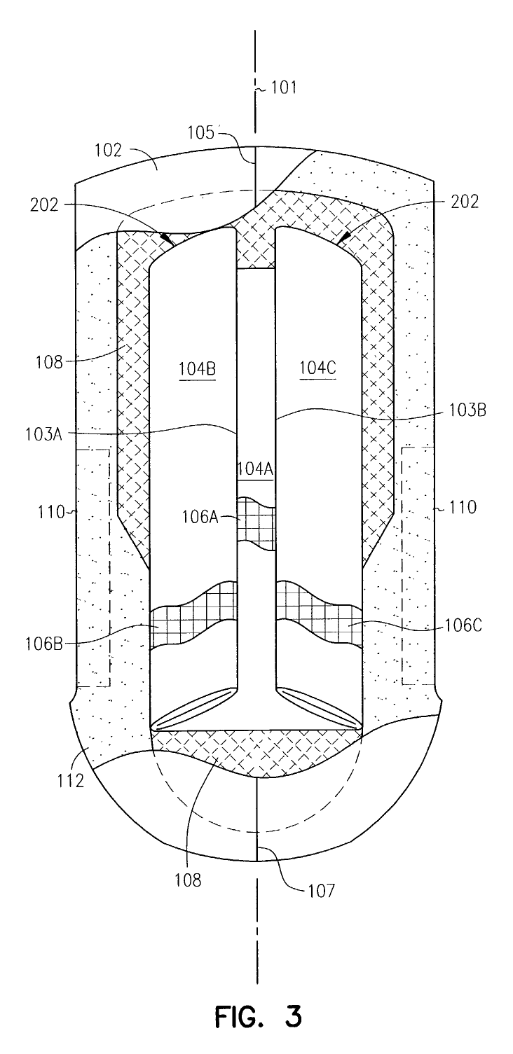

Referring again to FIG. 1, the liner 102, which is bonded to the cover 112 at its edges, essentially covers all surfaces of the various components described herein that may come in contact with a user's body. The liner 102 further has an opening created by a pair of folds having fold lines 103A and 103B, which are roughly parallel to a longitudinal centerline 101. Folded together with and contained within the folds of the liner 102 are most of the folded areas of the intake/distribution layer 104 and C-folded absorbent sheet 106. Specifically, the intake/distribution layer 104 is also folded generally along fold lines 103A and 103B to form an intake/distribution center section 104A and two intake/distribution side sections, 104B and 104C. The C-folded absorbent sheet 106 is C-folded, i.e., folded along its length in two different locations, both folds substantially parallel to the centerline 101, to form an absorbent sheet center section 106A and two absorbent sheet side sections 106B and 106C. As FIG. 1 shows, the centerline 101 runs lengthwise at approximately the midpoint of the center sections, 104A/106A. The side sections of the intake/distribution layer 104B/104C as well as the side sections of the C-folded absorbent sheet 106B/106C essentially form a pair of layered or composite longitudinal “flaps,” shown more clearly in FIGS. 2 and 3, i.e., flaps 202.

The fold lines 103A and 103B are optionally bonded together near the top and bottom of the article 100, along upper and lower seam lines, 105 and 107, respectively, i.e., in the area where the C-folded absorbent sheet 106 and intake/distribution layer 104 are not contained within the folds of the liner 102. In other words, only the end portions of the folds, i.e., the portions containing only the liner 102, are bonded together in this embodiment. In this way, some structure or support is provided for the components located between the liner 102 and cover 112, thus limiting excessive movement of these components during use. In one embodiment, the upper and lower seam lines, 105 and 107, respectively, have upper and lower bonding distances, 130 and 132, respectively, of between about 1.3 to five (5) cm (about 0.5 to two (2) in). In another embodiment, the upper and lower bonding distances, 130 and 132, respectively, are between about 2.5 and 3.8 cm (about one (1) and 1.5 in). The bonding distances 130 and 132 are not necessarily the same. In one embodiment, the upper bonding distance 130 is more than the lower bonding distance 132. In another embodiment, the upper bonding distance 130 is less than the lower bonding distance 132. In a specific embodiment, the upper bonding distance 130 is about 3.2 cm (1.25 in) and the lower bonding distance 132 is about 3.2 cm (1.5 in).

The side sections of the C-folded absorbent sheet 106 and intake/distribution layer 104 can be any suitable size and shape, although in most embodiments, each side section of the intake/distribution layer 104 is about the same size as the respective side section of the C-folded absorbent sheet 106 which it surrounds. In most embodiments, the side sections are substantially rectangular and about one-half the width of the respective center sections. In other words, the inner edge of each of the side sections 104B/104C and 106B/106C extend substantially to the centerline 101, with edges touching and/or nearly touching, such as in the embodiment shown in FIG. 1. In one embodiment, the side sections are each about 1.9 to 5.1 cm (about 0.75 to two (2) in) wide and about 17.8 to 23 cm (about 7 to 9 in) long, although, in the case of a child user, the size may be much less, down to about 1.3 cm (0.5 in) in width and about 7.6 to 15.2 cm (about 3 to 6 in) in length. In a particular embodiment, the side sections are each about 3.8 cm (about 1.5 in) wide and about 21.6 cm (about 8.5 in) long. In another embodiment, the side sections do not extend to the centerline 101. In yet another embodiment, the side sections are of variable width. In the embodiment shown in FIG. 1, the side sections each have a slightly variable width and do not extend to the centerline 101 throughout the entire length, leaving a gap of up to about 1.3 cm (about 0.5 in) in an area near the top and/or the bottom of the C-folded absorbent sheet 106 and intake/distribution layer 104 as shown. In one embodiment, the gap is not greater than about 0.6 cm (about 0.25 in). In one embodiment, the side sections overlap.

FIG. 1 shows the article 100 with the side sections 104B/106B and 104C/106C in a “closed” or “folded” position. The article 100 can be worn in this configuration, thus providing an additional layer of absorbent protection for the user. However, in the embodiment in which the side sections overlap, the article could be uncomfortable if worn when the side sections are in the closed position.

FIG. 2 shows an enlarged cross-sectional view of the article 100 with the side sections 104B/106B and 104C/106C in an “open” or “unfolded” position. The flaps 202 created by the side sections can also be seen, together with the fold lines 103A and 103B, as described above. It should be noted that although the combined folded layers, i.e., the liner 102, the intake/distribution layer 104 and the C-folded absorbent sheet 106, together have a C-folded cross section, it is only the C-folded absorbent sheet 106 that individually has a C-folded cross-section in this embodiment.

FIG. 3 provides a top view of the article 100 with the side sections 104B/106B and 104C/106C in the open position. The optional feature of the present invention, i.e., being able to wear the article with the side sections in either an open or closed position, allows the user to choose a preferred configuration of the components for his own personal needs. Specifically, if the user desires greater protection from leakage along the outside edges of the absorbent layers, the article can be worn with the side sections in an open position. In other words, the “B” side sections of the C-folded absorbent sheet and intake/distribution layer, 106B and 104B, respectively, can optionally be lifted, together with the surrounding liner 102, in a direction as shown by arrow 120 in FIG. 1 to form the containment flap 202, as shown in FIGS. 2 and 3. Similarly, the “C” side sections of the C-folded absorbent sheet and intake/distribution layer, 106C and 104C, can also optionally be lifted, together with the surrounding liner 102, in an opposing direction, as shown by arrow 122 shown in FIG. 1, to form another containment flap 202, as shown in FIGS. 2 and 3.

Referring again to FIG. 3, preferably the flaps 202 are sufficiently “tall” to provide adequate cupping protection during use in the open position. As discussed above, the side sections that form the flaps 202 can each have a width in a wide range of sizes, typically from about 1.3 to five (5) cm (about 0.5 to two (2) in). Again, the flaps 202 can be angled, curved, and so forth, essentially having a varied size from top to bottom.

As noted above, the article 100 includes other components known in the art, such as the cover 112, the garment-side absorbent layer 108 and the side elastics 110. The cover 112 defines the product's overall geometry, which can be any suitable size and shape. In most embodiments, the cover 112 is generally curved, extending beyond the absorbent components anywhere from about 0.6 to 2.5 cm (about 0.25 to one (1) in). In most embodiments, the cover 112 is about 23 to 33 cm (about 9 to 13 in) in length and about 12.7 to 17.8 cm (about 5 to 7 in) in width.

The garment-side absorbent layer 108 can be of any suitable size and shape as is known in the art for male incontinence articles. Preferably, the garment-side absorbent layer is located within the article's center area, i.e., target absorbency zone, to maximize absorbency. In the embodiment shown in FIGS. 1-4, the garment-side absorbent layer 108 has a paddle shape that includes a wide portion and a narrow portion as shown, although the invention is not so limited. Such a shape, however, has been found to provide significant protection in terms of leakage prevention, absorbency, coverage, and so forth, together with a high level of comfort. Specifically, the wide portion of the “paddle” is intended to be worn on the front side of a user to provide a larger absorbent area where needed by males, due to the shifting of the male genitalia during normal activity. The narrow section of the “paddle” is designed o fit more comfortably between the legs in the crotch area of the user. See, for example, U.S. Pat. Nos. D440,307 and D443,928, to Richardson, et al, both entitled “Absorbent Article,” commonly assigned, and hereby incorporated herein by reference, for an exemplary paddle shape.

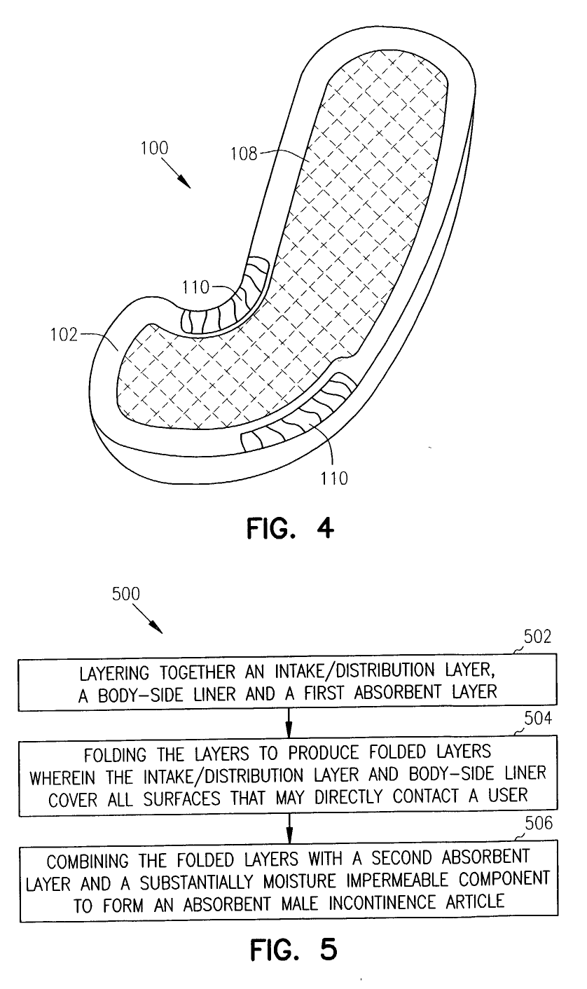

The side elastics 110 are positioned to cause the article 100 to cup along the sides in a lateral direction, as well as from front to back, i.e., in a longitudinal direction, essentially forming a “J” shape, as shown in FIG. 4. The side elastics 110 therefore help prevent leakage and improve product fit by causing the article 100 to better conform to the body. In embodiments in which the article 100 is secured to an undergarment, the side elastics 110 further help to conform the article 100 to the undergarment shape. The side elastics 110 can be made from any suitable materials, including, but not limited to, elastic strands of Lycra® made by DuPont, having offices in Wilmington, Del., or any type of stretch bonded laminate material, such as the material described in U.S. Pat. No. 4,720,415 to Vander Wielen et al, entitled, “Composite Elastomeric Material and Process for Making the Same,” commonly assigned, and hereby incorporated herein by reference.

Other materials known in the art can be also used for the various other components described herein. Referring again to FIGS. 1-3, the liner 102 is typically a thin, semitransparent material used to contain all the layers except the cover 112, to which it is secured along its edges, although the invention is not so limited. In one embodiment, the intake/distribution layer 104 and C-folded absorbent sheet 106 are not wrapped in any type of liner. In one embodiment, the liner 102 is made from a soft, flexible porous sheet that permits the passage of fluids therethrough, including, but not limited to, hydrophobic or hydrophilic nonwoven webs, wet strength papers, spunwoven filament sheets, and so forth. In one embodiment, the inner body-side surface is made from spunwoven polypropylene filaments with spot embossing, further including a perforated surface or suitable surfactant treatment to aid fluid transfer.

The absorbent components, including the C-folded absorbent sheet 106 and the garment-side absorbent layer 108, can be made from any material that absorbs exudates, including various liquids and/or fluids excreted or exuded by the user. For example, the absorbent components can be made of airformed, airlaid and/or wetlaid composites of fibers and high absorbency materials, referred to as superabsorbents. Superabsorbents typically are made of polyacrylic acids, such as DOW 2035 available from DOW Chemical of Midland, Mich. The fibers can be fluff pulp materials, such as Alliance CR-1654, or any combination of crosslinked pulps, hardwood, softwood, and synthetic fibers. Airlaid and wetlaid structures typically include binding agents, which are used to stabilize the structure. Other absorbent materials, alone or in combination, and including webs of carded or air-laid textile fibers, multiple plys of creped cellulose wadding, various super absorbent materials, various foams, such as synthetic foam sheets, absorbent films, and the like may also be used. The absorbent components may also be slightly compressed or embossed in selected areas as desired. Various acceptable absorbent materials are disclosed in U.S. Pat. No. 5,147,343, entitled, “Absorbent Products Containing Hydrogels With Ability To Swell Against Pressure,” U.S. Pat. No. 5,601,542, entitled “Absorbent Composite,” and U.S. Pat. No. 5,651,862, entitled, “Wet Formed Absorbent Composite,” all of which are commonly assigned and hereby incorporated herein by reference. Furthermore, the properties of high-absorbency particles can range from about zero (0) to about 100%, and the proportion of fibrous material from about zero (0) to about 100%.

In one embodiment, the absorbent components are made of fibrous absorbent materials with relatively high internal integrity, including, for example, one made with thermoplastic binder fibers in airlaid absorbents, e.g., pulp, bicomponent binding fibers, and superabsorbents, which have differing levels of densities. The higher density and resulting smaller capillary size in these regions promotes better wicking of the liquid. Better wicking, in turn, promotes higher utilization of the absorbent material and tends to result in more uniform swelling throughout the absorbent material as it absorbs the liquid.

In the embodiment shown in FIGS. 1-3, there are two layers of absorbent, namely the C-folded absorbent sheet 106 and the garment-side absorbent layer 108, which is in fluid communication with the C-folded absorbent sheet 106. Use of two different absorbent layers allows the product to have additional absorbent capacity in the area where the product needs to absorb the most fluid, i.e., the target area.

In a preferred embodiment, each absorbent layer, 106 and 108, has different absorbency levels, in order to enhance product performance. In one embodiment, the C-folded absorbent sheet 106 comprises a fibrous matrix containing high-absorbency material particles in a concentration greater than in the garment-side absorbent layer 108. In a specific embodiment, the C-folded absorbent sheet 106 has a lower average density and a greater basis weight than the garment-side absorbent layer 108. See, for example, U.S. Pat. No. 5,356, 403 to Faulks, et al., entitled “Absorbent Structure Comprising a Fibrous Matrix Having Dual Z-Directional Gradient,” commonly assigned, and hereby incorporated herein by reference.

The intake/distribution layer 104 typically has an operable level of density and basis weight designed to quickly collect and temporarily hold liquid surges, to transport the liquid from its initial entrance point and to substantially release the liquid to other parts of the absorbent article. Such a configuration can help prevent liquid from pooling and collecting on the portion of the absorbent article positioned against the wearer's skin, thereby reducing the feeling of wetness by the wearer.

Generally, the intake/distribution layer 104 is made from any suitable material to increase the weight of fluid intake retention. For example, the intake/distribution layer 104 can be a layer composed of a meltblown or spunbonded web of polyolefin fibers. Alternately, the intake/distribution layer 104 can be a bonded-carded-web or an airlaid web composed of natural and synthetic fibers. The bonded-carded-web may, for example, be a powder-bonded-carded web, an infrared bonded carded web, or a through-air-bonded-carded web. The infrared and through-air bonded carded webs can optionally include a mixture of different fibers and the fiber lengths within a selected fabric web may be within the range of 2.5 to 7.6 cm (about one (1) to three (3) in). The intake/distribution layer 104 may be composed of a substantially hydrophobic material that can optionally be treated with a surfactant or otherwise processed to impart a desired level of wettability and hydrophilicity. In one embodiment, the intake/distribution layer 104 is a meltspun material made by Kimberly-Clark Inc. of Neenah, Wis. In a particular embodiment, the intake/distribution layer 104 is blue in color to enhance consumer perception of absorbency, which has been established with various other absorbent products.

As noted above, the cover 112 serves as a fluid barrier and can be made from any suitable liquid impermeable material or a material treated to be liquid impermeable. In one embodiment, the cover 112 is a laminate comprised of an inner liner layer and an outer film layer, such as a polyethylene film. In one embodiment, “breathable stretch thermal laminate” (BSTL) is used for the cover 112. In an alternative embodiment the cover 112 is an opaque sheet of material with an embossed or matte surface that is about one mil thick, although the invention is not so limited. In another alternative embodiment, the outer surface is made of extensible materials, such as necked, pleated (or micropleated) or creped nonwovens, including spunbond polypropylenes, bonded carded webs, or laminates of nonwovens and films that are necked, pleated or creped so as to allow the outer cover to extend with minimal force. For example, a suitable extensible material is a 60% necked, polypropylene spunbound having a basis weight of about 1.2 osy. For a further description of extensible materials, see U.S. patent application Ser. No. 09,855,182, filed on May 14, 2001, entitled, “Absorbent Garment with Expandable Absorbent Element,” commonly assigned, and hereby incorporated herein by reference. The cover 112 can also be made of nonwovens, films, or composites of films and nonwovens.

The individual layers of the article 100 are combined into laminated layers using any suitable type of adhesive means known in the art that can maintain a bond during use. Such adhesives are generally tacky upon application to a first layer and remain so for an amount of time sufficient to allow for placement of the desired components onto the first layer. In one embodiment, the first layer is the cover 112 and the components placed on top of the adhesive applied to the cover 112 include side elastics 110, garment-side absorbent sheet 106, the outer side of the intake/distribution layer 104 and the outer portion of the liner 102. In one embodiment, an adhesive known as “H2717” manufactured by Bostik-Findley, Inc., having offices in Milwaukee, Wis., is used.

Other details of conventional construction and materials of disposable garments are understood in the art and will not be discussed in detail herein. See, for example, U.S. Pat. No. 4,437,860 to Sigl, commonly assigned, which is hereby incorporated herein by reference.

The invention further includes a method of making an incontinence article. As shown in FIG. 5, the process 500 begins by layering together 502 an intake/distribution, a body-side liner and a first absorbent layer. The process continues by folding 506 the layers to produce folded layers, wherein the intake/distribution layer and body-side liner cover all surfaces that may directly contact a user. The process continues by combining 510 the folded layers with a second absorbent layer and a substantially moisture impermeable component to form an absorbent male incontinence article. In one embodiment, the process includes bonding together the end portions of the folds. In one embodiment, the folded layers have a C-folded cross-section. In one embodiment some or all of the folded layers, such as the intake/distribution layer and first absorbent layer, are bonded together prior to being folded. In another embodiment, the first absorbent layer is wrapped in a tissue material to help maintain the integrity of the first absorbent layer during manufacturing. In one embodiment, the tissue material is made from any type of suitable wood pulp material and is similar in nature to single ply facial tissue. In one embodiment a folding board is used to create the folds. In one embodiment, the method further comprises applying adhesive and a removable strip to an outer side of the male incontinence absorbent article.

The product of the present invention is designed to be worn inside undergarments, and can be worn without any added means of securing it to the undergarment. However, in most embodiments, there are additional securing means, such as hook and loop securing means, tape, adhesive and the like. In one embodiment, some or all of the outer cover contains an adhesive coating that is covered by a protective paper, i.e., removable strip, prior to use. In such an embodiment, the user (or person assisting the user) peels off the removable strip and positions the product in the desired location inside an undergarment. Preferably, the product can be removed and repositioned or disposed of, as desired. In this way, the product can be secured to an undergarment, yet is easily removable.

The product of the present invention provides, in one embodiment, an absorbent article that provides soft and comfortable side shields made of absorbent material that help contain the male genitalia within the product's center or target absorbency zone, and absorb urine on all sides. Another advantage of the present invention is the optional nature of the above feature. Specifically, the user can choose instead to fold the side shields into the center of the product, thus providing an additional layer of absorbency.

All publications, patents, and patent documents cited in the specification are incorporated by reference herein, as though individually incorporated by reference. In the case of any inconsistencies, the present disclosure, including any definitions therein, will prevail.

Although specific aspects have been illustrated and described herein, it will be appreciated by those of ordinary skill in the art that any arrangement that is calculated to achieve the same purpose may be substituted for the specific aspect shown. This application is intended to cover any adaptations or variations of the present invention. For example, although the invention has been described primarily in terms of an article rather than an undergarment, the novel features of the present invention can also be incorporated into any type of male absorbent undergarment. Therefore, it is manifestly intended that this invention be limited only by the claims and the equivalents thereof.

What is claimed is:

1. An absorbent article comprising:
an absorbent sheet having a body-side and a garment-side, the absorbent sheet folded to form a central section and two side sections, each of the two side sections and central section having an inner surface and an outer surface; and
an intake/distribution layer covering the inner surface and the outer surface of each of the two side sections to form covered side sections, wherein the covered side sections form an opposed pair of longitudinal flaps.

2. The article of claim 1 wherein the absorbent sheet has a C-folded cross section.

3. The article of claim 2 wherein the intake/distribution layer covers the inner surface of the central section to form a covered central section.

4. The absorbent article of claim 3 wherein the opposed pair of longitudinal flaps are optionally unfolded into an open position during use.

5. The article of claim 4 wherein the longitudinal flaps serve as side shields in the unfolded position, further wherein the inner surface of each of the two side sections is the body-side.

6. The article of claim 3 wherein the longitudinal flaps are flat-folded during use.

7. The article of claim 6 wherein the longitudinal flaps serve as an additional absorbent layer substantially covering the covered central section when flat-folded, further wherein the outer surface of the two side sections is the body-side.

8. The article of claim 4 wherein the longitudinal flaps are about 1.3 to 5 cm in width.

9. The article of claim 3 further comprising:
a garment-side absorbent layer in fluid communication with the absorbent sheet and intake/distribution layer;
a substantially moisture impermeable garment-side outer cover adjacent to the garment-side absorbent layer, the garment-side outer cover having an outer surface; and
a moisture permeable body-side liner folded around the intake/distribution layer, the absorbent sheet and the garment-side absorbent layer, wherein the moisture permeable body-side liner forms a covering over the pair of longitudinal flaps.

10. The article of claim 9 further comprising a pair of side elastics contained between the body-side liner and the outer cover.

11. The article of claim 10 wherein the outer surface of the garment-side layer contains adhesive protected by a removable strip of paper.

12. The article of claim 9 wherein the liner has fold lines that extend beyond the longitudinal flaps.

13. The article of claim 12 wherein the fold lines are optionally bonded together at upper and lower ends of the article

14. The article of claim 13 wherein the fold lines are bonded together for a distance of about 1.3 to 5 cm.

15. An article comprising:
a moisture permeable body-side liner;
a dual-layer absorbent component adjacent to the moisture permeable body-side liner, the dual-layer absorbent component having a first layer and a second layer, the first layer having a body-side surface;
an intake-distribution layer surrounding at least a portion of the body-side surface of the first layer, the intake/distribution layer in fluid communication with the first and second layers of the dual-layer absorbent component; and
a substantially moisture impermeable garment-side cover adjacent to the intake-distribution layer.

16. The article of claim 15 wherein the first layer of the dual-layer absorbent component is a C-folded absorbent sheet.

17. The article of claim 16 wherein the second layer is a paddle-shaped garment-side absorbent layer.

18. The article of claim 17 further comprising side elastics sandwiched between outer edges of the body-side liner and the garment-side cover, wherein the side elastics cause the article to have a J-shaped configuration.

19. The article of claim 18 further comprising adhesive located on an outer surface of the garment-side cover, the adhesive protected by a removable strip of paper.

20. An absorbent article comprising:
a first absorbent layer having a body-side and a garment-side, the first absorbent layer folded to form a central section and two side sections, each of the two side sections and central section having an inner surface and an outer surface;
an intake/distribution layer covering the inner surface and the outer surface of each of the two side sections to form covered side sections, wherein the covered side sections form an opposed pair of longitudinal flaps; and
a second absorbent layer in fluid communication with the first absorbent layer.

21. The absorbent article of claim 20 wherein the absorbent article has an outer cover, further wherein the second absorbent layer is located between the garment-side of the first absorbent layer and the outer cover.

22. The absorbent article of claim 21 wherein the second absorbent layer has a paddle shape comprised of a wide section and a narrow section.

23. The absorbent article of claim 22 wherein the wide section is worn in front and the narrow portion is worn in a crotch area.

24. A method of making a male incontinence absorbent article comprising:
layering together an intake/distribution layer, a body-side liner and a first absorbent layer;
folding the intake/distribution layer, body-side liner and first absorbent layer to produce folded layers, wherein the intake/distribution layer and body-side liner cover all surfaces that may directly contact a user; and
combining the folded layers with a second absorbent layer and a substantially moisture impermeable component to form an absorbent male incontinence article.

25. The method of claim 24 wherein the body-side liner has folded end portions, further wherein the folded end portions are bonded together.

26. The method of claim 25 wherein the first absorbent layer has a C-folded cross-section after folding.

27. The method of claim 25 wherein the folded layers have a C-folded cross-section.

28. The method of claim 27 wherein the folded layers are created with a folding board.

29. The method of claim 27 wherein some or all of the folded layers are bonded together prior to being folded.

30. The method of claim 29 wherein the first absorbent layer and intake/distribution layer are bonded together prior to being folded.

31. The method of claim 27 wherein the first absorbent layer is wrapped in a tissue prior to being folded.

32. The method of claim 31 wherein the tissue is a wood pulp material.

33. The method of claim 27 further comprising adding a pair of side elastics to the male incontinence absorbent article.

34. The method of claim 33 further comprising applying adhesive and a removable strip to an outer side of the male incontinence absorbent article.
